(19) United States
(12) Patent Application Publication
(10) Pub. No.: US 2025/0170042 A1
(43) Pub. Date: May 29, 2025

(54) Method and Composition for Improving Skin Health

(71) Applicant: Lonza Greenwood LLC, Greenwood, SC (US)

(72) Inventors: Vijaya Juturu, Greenwood, SC (US); Zainulabedin Saiyed, Greenwood, SC (US)

(21) Appl. No.: 18/842,847

(22) PCT Filed: Mar. 2, 2023
(86) PCT No.: PCT/US2023/014353

§ 371 Date: Aug. 30, 2024

Related U.S. Application Data
(60) Provisional application No. 63/317,232, filed on Mar. 7, 2022.

(51) Int. Cl. A61K 8/65; A61K 8/27; A61K 8/67; A61Q 19/00; A61Q 19/08

(52) U.S. Cl. CPC A61K 8/65; A61K 8/27; A61K 8/676; A61K 8/678; A61Q 19/007; A61Q 19/08

(57) ABSTRACT

The present disclosure is directed to a method and composition for improving skin health of a mammal. The method includes administering a composition that includes a type II collagen and a collagen peptide to a healthy mammal.

### CROSS REFERENCE TO RELATED APPLICATIONS

This application claims priority to the benefit of U.S. Provisional Application No. 63/317,232, filed Mar. 7, 2022, which is expressly incorporated herein by reference in its entirety.

### BACKGROUND

In recent years, the use of collagen to treat various conditions has become exceedingly popular. Collagen is a protein that can be found in muscles, bones, skin, blood vessels, and in other parts of the body. There are various different types of collagen depending upon its function and form. For instance, Type I collagen, the most abundant collagen, is made of fibers found in tendons, ligaments, organs and skin. Type II collagen, on the other hand, primarily helps build cartilage, a major structural entity that sits on the surfaces of those bones which comprise articulating joints. Type III collagen is a major component of the extracellular matrix that makes up organs and skin. Type III collagen also forms blood vessels and tissue within the heart. Type IV collagen is found primarily in the skin as sheet-like structures in the cutaneous basal lamina.

Furthermore, Collagen peptides are portions of one or more of the a strands of any type of collagen formed through enzymatic hydrolysis of collagen. Collagen peptides are often used in beverages and food products, as they are water-soluble and non-gelling and have been used for joints.

Collagen can be found in numerous different products including cosmetic creams and body lotions. Collagen is also found in various oral supplements. Collagen production in the body, for instance, tends to slow as a person ages. Thus, collagen supplements have been taken in the past in order to reduce the effects of aging, by improving the health of skin and hair.

Collagen proteins and other denatured collagen generally require large doses to achieve benefits. It has been discovered that blending collagen peptides with an undenatured or native collagen source, the amount for the collagen peptide can be reduced to achieve the benefits of the collagen peptide for skin care applications. Thus, although collagen peptides can offer various advantages when administered to a human or animal, a need exists for a composition and method that can increase the effectiveness of collagen peptides and/or work in conjunction with collagen peptides to provide synergistic effects such improved efficacy or faster onset of action.

### SUMMARY

In one embodiment, the present disclosure relates to a method of improving skin health of a mammal, comprising administering a composition comprising a native or undenatured type II collagen and a collagen peptide to a healthy mammal in an amount sufficient to improve one or more of skin tone and elasticity, overall skin hydration, photoaging, skin microbiome, and wrinkle reduction. The type II collagen is present in the composition in an amount from about 1 mg to about 1000 mg; and wherein the collagen peptide is in the composition in an amount of 1 mg to about 10 g.

In another embodiment, the present disclosure relates to a composition for improving skin health of a mammal. The composition comprises a type II collagen present in an amount from about 1 mg to about 1000 mg; and a collagen peptide is present in the composition in an amount of 1 mg to about 10 g.

### DEFINITIONS

As used herein, the terms “about,” “approximately,” or “generally,” when used to modify a value, indicates that the value can be raised or lowered by 10% and remain within the disclosed aspect.

The term “therapeutically effective amount” as used herein, with, shall mean that dosage, or amount of a composition, that provides the specific pharmacological or nutritional response for which the composition is administered or delivered to mammals in need of such treatment. It is emphasized that “therapeutically effective amount”, administered to a particular subject in a particular instance, will not always be effective in treating the ailments or otherwise improve health as described herein, even though such dosage is deemed a “therapeutically effective amount” by those skilled in the art. Specific subjects may, in fact, be “refractory” to a “therapeutically effective amount”. For example, a refractory subject may have a low bioavailability or genetic variability in a specific receptor, a metabolic pathway, or a response capacity such that clinical efficacy is not obtainable. It is to be further understood that the composition, or supplement, in particular instances, can be measured as oral dosages, or with reference to ingredient levels that can be measured in blood. In other embodiments, dosages can be measured in amounts applied to the skin when the composition is contained with a topical formulation.

The term “supplement” means a product in addition to the normal diet of the mammal but may be combined with a mammal's normal food or drink composition. The supplement may be in any form but not limited to a solid, liquid, gel, capsule, or powder. A supplement may also be administered simultaneously with or as a component of a food composition which may comprise a food product, a beverage, a pet food, a snack, or a treat. In one embodiment, the beverage may be an activity drink.

The term “nutraceutical” and “ingredient” refers to any compound added to a dietary source (e.g., a fortified food or a dietary supplement) that provides health or medical benefits in addition to its basic nutritional value.

The term “delivering” or “administering” as used herein, refers to any route for providing the composition, product, or a nutraceutical, to a subject as accepted as standard by the medical community. For example, the present disclosure contemplates routes of delivering or administering that include oral ingestion plus any other suitable route of delivery including transdermal, intravenous, intraperitoneal, intramuscular, topical and subcutaneous.

As used herein, the term “collagen peptides” refers to fragments of collagen that are prepared from animal tissues. The collagen is produced from collagen found in the bones, skin, and connective tissue of animals using a hydrolysis. The process of hydrolysis typically involves breaking down the molecular bonds between individual collagen strands and peptides using combinations of physical, chemical or biological means.

As used herein, the term “mammal” includes any mammal that may benefit from improved skin health can include without limitation canine, equine, feline, bovine, ovine, human, or porcine mammals.

As used herein, “healthy” refers to the absence of illness or injury.

Other features and aspects of the present disclosure are discussed in greater detail below.

### DETAILED DESCRIPTION

It is to be understood by one of ordinary skill in the art that the present discussion is a description of exemplary embodiments only, and is not intended as limiting the broader aspects of the present disclosure.

The present disclosure is generally directed to a method of improving the skin health of a mammal. Particularly, the present disclosure has found that administering a composition that incudes type II collagen and collagen peptide, may unexpectedly improve the skin health of the mammal. For instance, in one aspect, the composition including a type II collagen, alone or in combination with another collagen source, and a collagen peptide, may inhibit oxidative stress, including ultraviolet oxidative stress, as well as improve hyperpigmentation from sun damage, age spots, and acne marks. Furthermore, the composition according to the present disclosure may increase skin elasticity, improve overall skin tone, smooth roughness, and reduce wrinkles. Furthermore, in one aspect, the composition of the present disclosure may also help to correct an imbalanced skin microbiome. The administration of a composition containing a type II collagen and a collagen peptide may also serve to decrease the amount of time or number of applications necessary to see any one or more of the above results. These results are surprising in light of the absence of any autoimmune condition and in light of the fact that skin damage has generally been considered to be the result of a physical or environmental stressor rather than an inflammatory.

Furthermore, in one aspect, a composition according to the present disclosure may be administered to a healthy mammal that may have an age that is at least 10%, such as least 15%, such as least 20%, such as least 25%, such as least 30%, such as least 35%, such as least 40%, such as least 45%, such as least 50%, such as least 55%, such as least 60%, such as least 65%, such as least 70%, such as least 75%, such as least 85%, such as least 90%, such as least 95% of its expected life span. The mammal may have an age such that it is less than about 95%, such as less than about 90%, such as less than about 85%, such as less than about 80%, such as less than about 75%, such as less than about 70%, such as less than about 65%, such as less than about 60%, such as less than about 55%, such as less than about 50%, such as less than about 45%, such as less than about 40%, such as less than about 35%, such as less than about 30%, such as less than about 25%, such as less than about 20%, such as less than about 15%, such as less than about 10% of its expected life span. A determination of life span may be based on actuarial tables, calculations, or the like. Nonetheless, in one aspect, the mammal may have an age that is less than about 65% of its expected life span, and may still exhibit the benefits discussed herein. Thus, it should be understood that, in one aspect, the composition according to the present disclosure may be administered to an ageing mammal that is healthy and therefore not suffering from any disease related decline in skin health.

Nonetheless, in one aspect, the composition according to the present disclosure may work together to provide greater benefit to cutaneous conditions that may be due to a chronic condition, or that occur in a healthy mammal, than the components alone. For instance, in one aspect, the composition including a type II collagen source and a collagen peptide may provide an anti-inflammatory effect, lessening skin redness and preventing further skin redness. Additionally or alternatively, the composition may work together to decrease fine lines and wrinkles, by improving the health of the existing skin and extending the length of life of skin cells, while also rebuilding tissue. Furthermore, the composition of the present disclosure may also serve to improve skin hydration, by improving the ability of the skin to take-up and retain moisture. Thus may also allow a greater firming effect to be observed. Therefore, the composition of the present disclosure may provide many benefits to skin, as well as greater benefits, than previously obtained.

In general, the supplement of the present disclosure contains a collagen source. The collagen source is a Type II collagen source, Type III collagen, Type IV collagen, or collagen peptides, or mixtures thereof. In one aspect, the collagen source contains type II collagen alone or in combination with one or more of Type I collagen, Type III collagen, or Type IV collagen. In one aspect, the collagen source may include a mixture of type II collagen (sometimes referred to as native type II collagen) and undenatured type II collagen. Additionally or alternatively, the collagen source may include a mixture of native type II collagen and undenatured type II collagen, in addition to a further collagen, such as Type I, Type III, or Type IV.

Nonetheless in one aspect, the collagen source includes an undenatured type II collagen, alone or in combination with native type II collagen. Particularly, the present disclosure has found that the collagen peptides and a collagen source including native and/or undenatured type II collagen, when combined, can increase the effectiveness of the resulting composition. In addition, or alternatively, the combination of a collagen source including native and/or undenatured type II collagen with a collagen peptide source can lessen the amount of time or number of applications necessary to see improvement to skin health.

As indicated above, the composition contains a collagen, particularly a Type II collagen such as an undenatured Type II collagen. Type II collagen for use in the present disclosure can be obtained from any suitable source. For instance, the collagen can be derived from a variety of mammalian sources, avian sources, or can be obtained from various fish species or a combination thereof. For instance, the collagen can be obtained from salmon, shark, poultry, porcine, eggshells, turkey cartilage, bovine cartilage, and the like. In one embodiment, for instance, the Type II collagen can be obtained as disclosed in U.S. Pat. No. 7,083,820 to Schilling which is incorporated by reference. For example, undenatured Type II collagen is available commercially as UC-II® from Lonza Greenwood LLC. UC-II® is a natural ingredient that contains a glycosylated, undenatured Type II collagen. The collagen source can also comprise a hydrolyzed collagen. The collagen source can also comprise a pure protein or active peptide fragments. In one embodiment, the collagen source can be free of any bone or bone material. In other embodiments, the collagen source can be free of any transforming growth factors (TGFs), bone morphogenetic proteins (BMPs), or both. In still another embodiment, the collagen source comprises Type II collagen and is completely free of any Type I collagen.

In preparing animal tissue for oral administration, in one embodiment, the Type Il collagen containing tissue can be first dissected free of surrounding tissues and diced or otherwise comminuted into particles. The particulate, or milled, cartilage can be sterilized by means which do not affect or denature the structure of a major portion of the Type II collagen in the tissue and formed into doses containing therapeutically effective levels of undenatured Type II collagen, said levels being generally in the amount of at least about 0.1 mg preferably from about 2 mg to about 500 mg of animal tissue in a dose. Being a natural product some variation from sample to sample is to be expected. These variations can be minimized by blending after comminution. The blending can be aided by analytical techniques which allow the measurement of the amount of undenatured Type II collagen and other constituents.

In one aspect, the total Type II collagen and/or total collagen source is present in the composition in an amount from about 1 milligram to about 5000 milligrams. For instance, the total Type II collagen and/or total collagen source can be present in the composition in an amount greater than about 1 milligrams, such as in an amount greater than about 10 milligrams, such as in an amount greater than about 15 milligrams, such as in an amount greater than about 20 milligrams, such as in an amount greater than about 25 milligrams, such as in an amount greater than about 30 milligrams. The total amount of Type II collagen and/or total collagen source present in the composition is generally be less than about 1000 milligrams, such as less than about 900 milligrams, such as less than about 800 milligrams, such as less than about 700 milligrams, such as less than about 600 milligrams, such as less than about 500 milligrams, such as less than about 400 milligrams, such as less than about 300 milligrams, such as less than about 200 milligrams, such as less than about 100 milligrams, such as less than about 80 milligrams, such as less than about 70 milligrams, such as less than about 60 milligrams, or any ranges or values therebetween. Additionally or alternatively, the total type II collagen and/or total collagen source may be present in the composition in an amount of about 30% to about 90% by weight, such as about 35% to about 85%, such as about 40% to about 80%, such as about 45% to about 75%, such as about 50% to about 70%, such as about 55% to about 65%, or any ranges or values therebetween.

In one aspect, the undenatured type II collagen may form all, or substantially all, of the total type II collagen, and therefore, may be present in the above discussed amounts. However, in one aspect, the undenatured type II collagen may account for about 1% to about 95% of the total type II collagen and/or total collagen source, such as about 2.5% to about 75%, such as about 5% to about 50%, such as about 10% to about 40% of the total type II collagen, or any ranges or values therebetween. Therefore, in one aspect, the undenatured type II collagen may be present in the composition in an amount of 0.1 mg to about 100 mg, such as about 0.5 mg to about 75 mg, such as about 0.75 mg to about 50 mg, such as about 1 mg to about 30 mg, or any ranges or values therebetween.

Furthermore, in one aspect, the type II collagen and/or collagen source may further include a preservative salt, such as potassium chloride. Thus, in one aspect, the total amounts of type II collagen or total collagen source discussed above may include type II collagen and/or undenatured type II collagen, alone or in combination with a further collagen, a preservative salt, or combinations thereof. In such as aspect, the total type II collagen, including native and undenatured type II collagen, may account for about 1% to about 99% of the total collagen source, such as about 2.5% to about 90%, such as about 5% to about 80%, such as about 7.5% to about 70%, such as about 10% to about 60%, such as about 15% to about 50%, such as about 20% to about 35%, or any ranges or values therebetween. Thus, in one aspect, the total amount of type II collagen, including native and undenatured type II collagen in the composition may be from about 1 mg to about 1000 mg, such as about 2.5 mg to about 500 mg, such as about 5 mg to about 250 mg, such as about 7.5 mg to about 100 mg, such as about 10 mg to about 40 mg, or any ranges or values therebetween. Of course, in one aspect, no preservative salt is used.

Moreover, in one aspect, when the type II collagen includes undenatured type II collagen, the undenatured type II collagen may have a large oxygen radical absorbance capacity (ORAC), as measured according to ORAC 6.0. Particularly, ORAC tests measure antioxidant scavenging activity against oxygen radicals that are known to be involved in the pathogensis of aging and common disease, and consist of six types of ORAC assays that evaluate the antioxidant capacity of a material against primary reactive oxygen species, peroxyl radical, hydroxyl radical, superoxide anion, and peroxynitrite. Particularly, the ORAC assay includes introducing a reactive oxygen species (ROS) introducer to the assay system, where the ROS introducer triggers the release of a specific ROS which would degrade the probe and cause its emission wavelength or intensity to change. Thus, if the assay being tested includes an antioxidant, the antioxidant absorbs the ROS and preserves the probe from degradation. The degree of probe preservation indicates the antioxidant capacity of the material, and the results are expressed as μmol trolox equivalents (TE)/g of a tested material.

For example, an ORAC assay against peroxyl radical measures the antioxidant capacity of a sample to protect the fluorescent protein (fluorescein) from damage by a peroxyl radical which is generated from 2,2′azobis(2 amidinopropane) dihydrochloride (AAPH). The ORAC assay against hydroxyl radical measures the antioxidant capacity of the sample to protect the fluorescent protein (fluorescein) from damage by a hydroxyl radical which is generated from reaction between cobalt and hydrogen peroxide. The ORAC assay against peroxynitrite measures the antioxidant capacity of the sample to protect Dihydrorhodamine-123 from damage by a peroxynitrite radical which is generated from 3-morpholinosyndnonimine hydrochloride. The ORAC assay against superoxide measures the antioxidant capacity of the sample to protect hydroethidine from damage by a superoxide which is generated from xanthine oxidase. The ORAC assay against singlet oxygen measures the antioxidant capacity of the sample to protect hydroethidine from damage by single oxygen which is generated from a reaction between lithium molybdate and hydrogen peroxide. Finally, the ORAC assay against hypochlorite measures the antioxidant capacity of the sample to protect the fluorescent protein fluorescein from damage by the hypochlorite radical which is generated from sodium hypochlorite.

Thus, in one aspect, an undenatured type II collagen according to the present disclosure may have a total ORAC of about 200 μmol TE/g or greater, such as about 250 μmol TE/g or greater, such as about 300 μmol TE/g or greater, such as about 350 μmol TE/g or greater, such as about 400 μmol TE/g or greater, such as about 450 μmol TE/g or greater, such as about 500 μmol TE/g or greater, such as about 550 μmol TE/g or greater, such as about 600 μmol TE/g or greater, such as about 700 μmol TE/g or greater, such as about 750 μmol TE/g or greater, such as about 800 μmol TE/g or greater, such as about 825 μmol TE/g or greater, up to about 1000 μmol TE/g, or any ranges or values therebetween.

Furthermore, in one aspect, an undenatured type II collagen according to the present disclosure may have a ORAC against peroxyl radicals of about 1 μmol TE/g or greater, such as about 2.5 μmol TE/g or greater, such as about 5 μmol TE/g or greater, such as about 7.5 μmol TE/g or greater, such as about 10 μmol TE/g or greater, such as up to about 10.5 μmol TE/g or greater, up to about 50 μmol TE/g, or any ranges or values therebetween.

Similarly, in one aspect, an undenatured type II collagen according to the present disclosure may have a ORAC against hydroxyl radicals of about 10 μmol TE/g or greater, such as about 15 μmol TE/g or greater, such as about 20 μmol TE/g or greater, such as about 25 μmol TE/g or greater, such as about 27.5 μmol TE/g or greater, such as about 30 μmol TE/g or greater, up to about 40 μmol TE/g, or any ranges or values therebetween.

Additionally or alternatively, in one aspect, an undenatured type II collagen according to the present disclosure may have a ORAC against peroxynitrite of about 0.5 μmol TE/g or greater, such as about 1 μmol TE/g or greater, such as about 1.5 μmol TE/g or greater, such as about 2 μmol TE/g or greater, such as about 2.25 μmol TE/g or greater, up to about 5 μmol TE/g, or any ranges or values therebetween.

In one aspect, an undenatured type II collagen according to the present disclosure may have a ORAC against singlet oxygen of about 500 μmol TE/g or greater, such as about 550 μmol TE/g or greater, such as about 600 μmol TE/g or greater, such as about 650 μmol TE/g or greater, such as about 700 μmol TE/g or greater, such as about 725 μmol TE/g or greater, up to about 1000 μmol TE/g, or any ranges or values therebetween.

Furthermore, in one aspect, an undenatured type II collagen according to the present disclosure may have a ORAC against hypochlorite of about 25 μmol TE/g or greater, such as about 30 μmol TE/g or greater, such as about 35 μmol TE/g or greater, such as about 40 μmol TE/g or greater, such as about 45 μmol TE/g or greater, such as up to about 50 μmol TE/g or greater, up to about 75 μmol TE/g, or any ranges or values therebetween.

Additionally or alternatively, as discussed above, the collagen source is combined with a collagen peptide. The term “collagen peptide” (CP) as used herein refers to a peptide derived from collagen, collagen peptides are short chains of amino acids typically extracted from native (full-length) collagen such as via enzymatic hydrolysis (also called enzymatic hydrolysation). The degree of hydrolysis usually has an impact on the average molecular weight of the final product. The term “collagen peptide” may be used interchangeably and synonymous with the terms “hydrolyzed collagen” or “collagen hydrolysate”.

In a one embodiment, the collagen peptide as part of the current invention is derived from collagen obtained from animal skin, cartilage, bone, and/or connective tissue. In another embodiment, the collagen peptide as part of the current invention is derived from porcine, bovine and/or fish collagen. The collagen as disclosed herein is preferably derived from collagen type I and/or collagen type II.

The collagen peptide as taught in the current invention may comprise collagen which is hydrolyzed or partially hydrolyzed, wherein the collagen may be derived from any species of animal. In addition or alternatively, an animal herein may refer to any animal capable of providing connective tissue, and the connective tissue is used to prepare collagen peptide.

In one embodiment, the collagen as taught herein is derived from a cow. In another embodiment, the collagen as taught herein is derived from a pig. In yet another embodiment, the collagen as taught herein is derived from a fish.

In various embodiments, the collagen as provided in the combination of the current invention is a mixture of collagen from different sources, such as collagen originating from multiple animal species, and/or collagen originating from different tissues. For example, the collagen as provided in the combination of the current invention may be a mixture of collagen chosen from fish collagen, porcine collagen, and bovine collagen. In addition or alternatively, the collagen may be a mixture of collagen chosen from skin-, cartilage-, bone-, and/or connective tissue-derived collagen. The connective tissue as taught herein refers to a group of tissues present between various tissues, organs and cells, including but not limited to being present in the corpus callosum, skin, spines (e.g. antler), protrusions (e.g. humps, lion's milk), homs, head, brain, neck, ear, eye, nose, tongue, lip, mouth, esophagus, trachea, limbs, feet, toes, palms, claws, bones, cartilage, bone marrow, joints, membranes, hind, ligaments, tendons, tendons interval, tendon, rib, diaphragm, muscle, skeletal muscle, smooth muscle, intestine, esophagus, trachea, Venetian, belly, blood vessels, bladder, stomach, aorta, heart, liver, kidney, chest, lung, spleen, pancreas, egg, sperm, testis, ovary, nerve, gallbladder, belly.

In a particular embodiment, the collagen as taught herein is derived from the skin and/or skin connective tissue. In another preferred embodiment, the collagen as taught herein is derived from the hind. In another preferred embodiment, the collagen as taught herein is derived from cartilage. In yet another preferred embodiment, the collagen as taught herein is derived from bone.

In various embodiments of the current invention, the collagen peptide may be produced by the enzymatic hydrolysis or partial enzymatic hydrolysis of collagen, wherein the enzyme used for this purpose may be one or more selected from the group consisting of serine protease, alkaline protease, neutral protease, flavour protease, complex protease, thiol protease, bromelain, metalloprotease, aspartame, protease, carboxypeptidase, pepsin, chymotrypsin, trypsin, cathepsin K, chymotrypsin, papain, and subtilisin combination.

In a one embodiment of the present disclosure, the collagen peptide has a mean molecular weight (Mw) between 300 Da and 10000 Da, e.g. between 500 Da and 7500 Da, between 1000 Da and 6000 Da, between 1500 Da and 5000 Da, or between 1500 Da and 3000 Da. The molecular weight as disclosed herein is generally the weight averaged molecular weight Mw. In a more embodiment of the current invention, the collagen peptide has a mean molecular weight between 1500 Da and 3000 Da or a mean molecular weight between 3000 Da and 6000 Da.

The collagen peptide as taught herein may have a protein content of 75-99 wt. %, preferably 80-99 wt. %, more preferably 85-99 wt. %, most preferably 90-99 wt %. In an embodiment, the collagen peptide as part of the combination as taught herein comprises glycine in an amount of 15-25 wt. %, proline in an amount of 9-13 wt. %, and hydroxy proline in an amount of 9-13 wt. %, all per total weight of the amino acids in the collagen peptide.

In an embodiment, the collagen peptide as part of the combination as taught herein comprises glycine in an amount of 5-50 wt. %, preferably in an amount of 10-30 wt. %, more preferably in an amount of 12.5-27.5 wt. %, even more preferably in an amount of 15-25 wt. %, most preferably in an amount of 17.5-22.5 wt. %, all per total weight of the amino acids in the collagen peptide.

In a particular embodiment, the collagen peptide as part of the combination as taught herein comprises proline in an amount of 2-20 wt. %, preferably in an amount of 7-15 wt. %, more preferably in an amount of 8-14 wt. %, even more preferably in an amount of 9-13 wt. %, most preferably in an amount of 10-12 wt. %, all per total weight of the amino acids in the collagen peptide.

In another embodiment, the collagen peptide as part of the combination as taught herein comprises hydroxyproline in an amount of 2-20 wt. %, preferably in an amount of 7-15 wt. %, more preferably in an amount of 8-14 wt. %, even more preferably in an amount of 9-13 wt. %, most preferably in an amount of 10-12 wt %, all per total weight of the amino acids in the collagen peptide.

In a particular embodiment, the collagen peptide as part of the combination as taught herein has a molecular weight of 1750-2250 Da and comprises glycine in an amount of 17.5-22.5 wt. %, proline in an amount of 10-12 wt. %, and hydroxyproline in an amount of 10-12 wt %, all per total weight of the amino acids in the collagen peptide.

In another embodiment, the collagen peptide as part of the combination as taught herein has a molecular weight of 4750-5250 Da and comprises glycine in an amount of 17.5-22.5 wt. %, proline in an amount of 10-12 g wt. %, and hydroxyproline in an amount of 10-12 g wt. %, all per total weight of the amino acids in the collagen peptide.

In another exemplary embodiment, the collagen peptide as part of the combination as taught herein has a molecular weight of between 1500-3000 Da and comprises glycine in an amount of 17.5-22.5 wt. %, proline in an amount of 10-12 wt. %, and hydroxyproline in an amount of 10-12 wt. %, all per total weight of the amino acids in the collagen peptide.

In an embodiment, the collagen peptide is present in the combination in an amount of between 1 mg and 10,000 mg (10g), typically between 10 mg and 5,000 mg (5 g), more typically between 50 mg and 2500 mg, most typically 100 mg and 1000 mg, wherein the amount is the dry weight of the composition.

In addition to the collagen source and the collagen peptides, the composition may contain other beneficial components. For instance, the composition may contain one or more vitamins, minerals, essential fatty acids, amino acids, polyphenols stilbenoids, curcumininoids, tannins, flavones, flavonols, flavan-3-ols, flavanones, anthocyanidins, anthocyanins, isoflavones, flavanonols, proanthocyanidins, dihydroxybenzoic acids, carotenoids, and pyridine alkaloids. In one aspect, the composition may contain an effective amount of an antioxidant, an amino acid, an essential fatty acid, a polyphenol, or combinations thereof.

For example, in one aspect the composition contains at least one vitamin, such as at least one of vitamin B, vitamin C, and vitamin E. Vitamins may be contained in the composition in an amount of from about 50 μg/g of supplement to about 5000 μg/g, such as about 100 μg/g to about 4500, such as about 250 μg/g to about 4000 μg/g, such as about 400 μg/g to about 3500 μg/g, or any ranges or values therebetween. The above ranges may be for any one vitamin alone or a total amount of all vitamins. In one aspect, vitamin E is present in the composition in an amount of about 100 μg/g to about 1000 μg/g, such as about 250 μg/g to about 750 μg/g, such as about 400 μg/g to about 600 μg/g, or any ranges or values therebetween. In another aspect, vitamin C is present in the composition in an amount of about 1000 μg/g to about 5000 μg/g, such as about 2000 μg/g to about 4000 μg/g, such as about 3000 μg/g to about 3750 μg/g, or any ranges or values therebetween.

Furthermore, in an aspect, the composition contains at least one mineral, such as at least one of potassium magnesium, zinc, or calcium. Minerals may be contained in the composition in an amount of from about 1 mg/g to about 50 mg/g, such as about 2.5 mg/g to about 45 mg/g, such as about 5 mg/g to about 40 mg/g, or any ranges or values therebetween. The above ranges may be for any one mineral or a total amount of one mineral. In one aspect, the composition contains potassium in an amount of about 9.5 mg/g to about 12 mg/g, such as about 9.75 mg/g to about 11.5 mg/g, such as about 10 mg/g to about 11 mg/g, or any ranges or values therebetween. Similarly, in one aspect, the composition contains magnesium in an amount of about 1 mg/g to about 10 mg/g, such as about 2.5 mg/g to about 7.5 mg/g, such as about 4 mg/g to about 6 mg/g, or any ranges or values therebetween. Furthermore, in one aspect, the composition contains calcium in an amount of about 1 mg/g to about 50 mg/g, such as about 2.5 mg/g to about 47.5 mg/g, such as about 5 mg/g to about 45 mg/g, such as about 10 mg/g to ab out 40 mg/g, such as about 20 mg/g to about 37.5 mg/g, such as about 30 mg/g to about 35 mg/g, or any ranges or values therebetween.

Additionally, the composition may further include at least one additive that enhances sports performance or that further contributes to reducing oxidative stress. For instance, in one aspect, an additive may be one or more of curcumin, spirulina, astaxanthin, a superoxide dismutase (SOD) enriched supplement or carotenoids. Furthermore, in one aspect, the present disclosure may include one or more microalgae with high SOD and ORAC levels, where the one or more microalgae are different than a microalgae selected as the SOD enriched supplement. Particularly, such microalgae may further help to reduce oxidative stress, and may contribute further anti-inflammatory properties and protection against infections, including improvement in immune health. One such SOD enriched supplement includes the OCEANIX® product available from Lonza Greenwood LLC. Moreover, in another aspect, an additive may include one or more probiotics.

In one aspect, the composition is administered once per day, twice per day, three times per day, up to about five times per day. However, in one aspect, the composition is administered once per day.

Notwithstanding the final amounts of collagen, collagen peptide, and other additives contained in the composition, or delivery method used in one aspect, a mammal being administered the composition may exhibit an improvement in skin health as evidenced by a reduction in wrinkles of about 5% or greater, such as about 10% or greater, such as about 15% or greater, such as about 25% or greater, as measured or evaluated using image analysis, such as Visia CR, Primos, or QuanifiCare.

Furthermore, in one aspect, the mammal being administered the composition may exhibit an improvement in skin health as evidenced by a reduction in skin irregularities of about 5% or greater, such as about 10% or greater, such as about 15% or greater, such as about 25% or greater, as evaluated or measured by image analysis as discussed above. In one aspect, skin irregularities may be one or more of roughness, such as bumps, age spots, sun spots, sun damage, and skin discoloration or unevenness in coloring.

Moreover, in one aspect, the mammal being administered the composition may exhibit an improvement in skin health as evidenced by an improvement in skin flaccidness of about 5% or greater, such as about 10% or greater, such as about 15% or greater, such as about 25% or greater, as measured by image analysis as discussed above. For instance, in one aspect, a v-shape analysis may be used where a contour is drawn along the subjects jaw. The straightness of the line (e.g. no sagging around jowls) is analyzed and compared.

In yet a further aspect, the mammal being administered the composition may exhibit an improvement in skin health as evidenced by an improvement in skin moisture of about 5% or greater, such as about 10% or greater, such as about 15% or greater, such as about 25% or greater, as measured using electrical capacitance. For instance, in one aspect, the capacitance of a subjects skin cells may change due to the amount of moisture retained in the cells, and the change may be recorded.

While various aspects and benefits have been discussed, in one aspect, the composition is incorporated into a suitable delivery form prior to incorporation into a dosage form as discussed below. In one aspect, the composition may be coated onto a seed in order to form a multi-particulate dosage form that may be used in one or more of the administrations discussed in greater detail below. For instance, these multi-particulates may contain a starter particle or pellet, also known as seeds, beads, nonpareils, micro-granules, or starter cores. The cores are formed of inert material such as sucrose or microcrystalline cellulose. These cores are used as starter material and layered with active ingredient compounds before being incorporated into a final oral dosage formulation.

Nonetheless, in one aspect, the delivery form may include a lipid multiparticulate (LMP). For instance, such an aspect may include one or more particles, wherein each of the particles includes a core containing a lipid matrix having the composition dispersed therein, as the composition may be a fat-soluble active ingredient. The plurality of particles may also include one or more outer layers disposed thereon that may include one or more active ingredients, which may include the collagen source or the collagen peptide source, or which may be a further active ingredient. The plurality of particles may be incorporated into a capsule or any other suitable oral or other dosage form discussed in greater detail below.

In one aspect utilizing a LMP delivery, the lipid matrix of the core may be formulated such that the core contains from about 10% to about 60% by volume of the composition. For example, in certain embodiments, the core may contain at least about 15% by volume of the composition, such as at least about 20% by volume, such as at least about 25% by volume, such as at least about 30% by volume, such as at least about 35% by volume, such as at least about 40% by volume, such as at least about 45% by volume, such as at least about 50% by volume, or any ranges or values therebetween.

In certain aspects, the lipid matrix of the core may include a) at least one low flow point excipient, b) at least one high flow point excipient, c) at least one low-flow point surfactant, and c) optionally an antioxidant.

The cores disclosed herein may include a lipid matrix that contains a low-flow point excipient. For example, in certain embodiments the lipid matrix may contain one or more low-flow point excipients. Low flow point excipients generally include fatty alcohols, fatty acids, fatty acid esters of glycols and poly glycols, fatty acid esters of polyglycerol and fatty acid esters of glycerol (glycerides) with flow points of less than 50° C. When the low flow point excipient is a relatively pure material, the melting point is also less than 50° C. A preferred class of low flow point excipients are low flow point glycerides. By “low flow point” excipient, such as a glyceride, is meant that the melting point of the excipient, such as a glyceride, is less than 50° C. In some embodiments, the low flow point glyceride has a melting point of less than 40° C. In some embodiments, the low-flow point excipient, such as glyceride, is a mixture of compounds, having a flow point of 50° C. or less. In some embodiments, the low-flow point excipient, such as glyceride, has a flow point of 40° C. or less. In some embodiments, the low-flow point glyceride has a low flow point of 30° C. or less. Exemplary low flow point glycerides include polyglycolized glycerides, such as some of the Gelucire products manufactured by Gattefosse, such as Gelucire® 43/01 having a nominal melting point of 43° C. Mixtures of low flow point glycerides are also effective, such as mixtures of Gelucire® 43/01 (C10-C18 triglycerides), Gelucire® 50/13 (stearoyl polyoxylglycerides), Gelucire® 44/14 (lauroyl macrogol-32 glycerides), and mixtures thereof. Other glycerides may also be used, such as fatty acid esters of glycols and poly glycols, and fatty acid esters of polyglycerols.

A function of the low flow point excipient is to ensure that at least a significant portion of the formulation matrix softens when ingested orally by a patient in need of therapy, at the temperature of the GI tract (about 37 for humans). This allows the formulation to break down by digestion in the gastro-intestinal (GI) tract, and ultimately to disperse in the GI tract to promote dissolution and absorption of the active. In certain embodiments the low flow point excipient provides a significant portion of the formulation matrix to be present in a non-crystalline liquid or amorphous state when ingested and softened in the GI tract.

Exemplary low flow point fatty alcohols include myristyl alcohol (Tm 38° C.)., lauryl alcohol (Tm 23° C.) and capric alcohol (Tm 7° C.). Exemplary low flow point fatty acids include lauric acid (Tm 44° C.) and oleic acid (Tm 16° C.).

In certain aspects, the cores may include a lipid matrix including a high-flow point excipient. For example, in certain embodiments the lipid matrix may contain one or more high-flow point excipients. By “high flow point” excipient is meant an excipient that has a flow point 50° C. or more. High flow point excipients may also have a melting point above 50° C. High flow point excipients generally include fatty alcohols, fatty acids, fatty acid esters of glycols and poly glycols, fatty acid esters of polyglycerol, fatty acid esters of glycerol (glycerides), waxes, polar waxes and other materials with flow points of greater than 50. A preferred class of high flow point excipients are “high flow point glycerides”. By high flow point glyceride is meant that the flow point or melting point of the glyceride is 50° C. or more. In some embodiments, the high flow point glyceride has a melting point of 60° C. or more. In some embodiments, the high-melting point glyceride is a mixture of compounds, having a flow point of 50° C. or more. In some aspects, the high-flow point glyceride has a flow point of 60° C. or more. In some aspects, the high flow point glyceride has a flow point of 70° C. or more.

Exemplary high flow point glycerides include glycerol behenate, glycerol dibehenate, glycerol palmitate, hydrogenated castor oil, and mixtures thereof. Often, the high flow point glyceride is a mixture of compounds that are formulated into a product and sold under a variety of trade names. Furthermore, exemplary high flow point and high melt point fatty alcohols include stearyl alcohol (Tm 58° C.) and behenyl alcohol (Tm 71° C.). Exemplary high flow point and high melt point fatty acids include palmitic acid (Tm 63° C.) and stearic acid (Tm >70° C.). Additionally or alternatively, exemplary waxes include paraffin wax, beeswax, candelilla wax, carnauba wax, and mixtures thereof.

A function of the high flow point excipient is to aid in the manufacturability of the cores by enabling the cores to congeal at a lower temperature to obtain solid particles during the melt-spray-congeal processing. In certain aspects the high flow point excipient aids the physical stability of the core formulation. In most embodiments, the high flow point excipient is not appreciably digested in the GI tract.

In some aspects, the cores or the lipid matrix of the cores may include other excipients to improve the performance and chemical stability of the formulations. In one aspect, a dispersing agent is included in the core. Exemplary dispersing agents include lecithin, glycerin monostearate, ethylene glycol palmitostearate, aluminum oxide, polyethylene alky ethers, sorbitan esters, and mixtures thereof. In one aspect, the cores include an antioxidant to maintain chemical stability of the active agent. Exemplary antioxidants include vitamin E, tocopheryl polyethylene glycol succinate (TPGS), rosemary extract, ascorbic acid, asorbyl palmitate, butylated hydroxyanisole (BHA), buytlated hydroxytoluene (BHT), and mixtures and combinations thereof.

Furthermore, in one aspect, a flow aid is used to improve the flow properties of the cores. Exemplary flow aids also known as glidants include calcium silicate, cab-o-sil, silicon dioxide, calcium phosphate tribasic, colloidal silicone dioxide, magnesium silicate, magnesium trisilicate, starch, talc, and other flow aids.

The cores described herein are generally a plurality of particles or beadlets that are solid at ambient temperature and are generally spherical in shape. By generally spherical is meant that while most particles are essentially spherical, they do not necessarily form “perfect” spheres. Such particle variations in spherical shapes are known to those persons of ordinary skill in the art of melt-spray-congeal processing and similar particulate forming methods.

The cores may have a size ranging from a mean diameter of about 40 μm to about 3000 μm, such as from about 50 μm to about 2500 μm, such as from about 80 μm to about 2000 μm, such as from about 100 μm to about 1500 μm, such as from about 200 μm to about 1000 μm, such as from about 300 μm to about 800 μm. To measure the diameters of the particulates, there are several methods that can be used, including laser diffraction, optical microscopy, and/or SEM.

In certain aspects, the cores containing the active ingredient and lipid matrix have a flow point above 25° C., such as above 30° C., such as above 35° C., such as above 40° C.

In one aspect, the lipid matrix may contain fatty alcohols, fatty acids, fatty acid esters of glycerol, glycols and poly glycols, fatty acid esters of polyglycerol, polyglycolized glycerides, C8-C18 triglycerides, stearoyl polyoxylglycerides, lauroyl macrogol-32 glycerides, caprylocaproyl macrogol-8 glycerides, oleoyl macrogol-6 glycerides, linoleoyl macrogol-6 glycerides, myristyl alcohol, lauryl alcohol, capric alcohol, glycerol behenate, glycerol dibehenate, glycerol palmitate, hydrogenated castor oil, stearyl alcohol, behenyl alcohol, palmitic acid, stearic acid, paraffin wax, beeswax, candelilla wax, carnauba wax, polyethoxylated 12-hydroxysteric acid, propylene glycol monocaprylate esters, propylene glycol dicaprate/dicaprylate esters, propylene glycol heptanoate, propylene glycol monostearate, propoylene glycol monooleate, propylene glycol monopalmitate, propylene glycol monomyristate, esterified alpha-tocopheryl polyethylene glycol succinate, propylene glycol monolaurate esters, polyoxyl 35 castor oil, polyoxyl 40 hydrogenated castor oil, lecithins, vitamin E, tocopheryl polyethylene glycol succinate (TPGS), sugar fatty acid esters, sorbitan fatty acid esters, polyoxyethylene sorbitan fatty acid esters, polyoxyethylene-polyoxypropylene copolymers, propylene glycol, triacetin, isorpropyl myristate, diethylene glycol monoethyl ether, polyethylene glycol, glycerol, rosemary extract, ascorbic acid, ascorbyl palmitate, butylated hydroxyanisole (BHA), butylated hydroxytoluene (BHT), and mixtures and combinations thereof.

In a further aspect, the lipid matrix composition comprises greater than 50 wt % of the low flow point excipient. In one aspect, the lipid matrix composition comprises at least 2 wt % of the high flow point excipient. In yet a further aspect, the lipid matrix composition comprises less than 30 wt % of the high flow point excipient. Moreover, in one aspect, the mass ratio of the low flow excipient to the high flow excipient is at least 20:1, such as at least 15:1, such as at least 10:1, such as at least 4:1. such as at least 3:1, such as at least 2:1, or any ranges or values therebetween.

In one aspect, the composition is contained within a lipid matrix containing stearyl alcohol, stearic acid, candelilla wax, and lecithin. In one aspect the lipid matrix may contain from about 40% about 60% by weight of the active ingredient, such as about 50% by weight of the active ingredient. Furthermore, in an aspect, the lipid matrix may contain from about 15% to about 25% by weight of stearyl alcohol, such as about 17% by weight of stearyl alcohol. Additionally or alternatively, in an aspect, the lipid matrix may contain from about 10% to about 20% by weight of stearic acid, such as from about 15% by weight of stearic acid. In one aspect, the lipid matrix may contain from about 10% to about 20% of a suitable wax, such as candelilla wax, such as from about 15% by weight of a suitable wax. Moreover, in one aspect, the lipid matrix may contain from about 1% to about 3% of a lecithin, such as about 2% of a lecithin, such as soy lecithin.

In certain aspects, the lipid matrix containing the active ingredient may be formed and then formulated into one or more particles having a generally spherical shape and a mean diameter ranging from about ranging from 40 μm to 3000 μm, such as from about 100 μm to 2000 μm, such as from about 300 μm to 1000 μm.

In one aspect, the lipid matrix is comprised of at least 10 wt % to 50 wt % of the low flow point excipient. In another aspect, the lipid matrix is comprised of at least 50 wt % to 75 wt % of the low flow point excipient.

In one aspect, the lipid matrix comprises at least 2 wt % of the high flow point excipient. In another aspect, the lipid matrix is comprised of about 1 wt % to about 30 wt % of the high flow point excipient, such as about 2 wt % to about 20 wt % of the high flow point excipient, such as about 3 wt % to about 15 wt % of the high flow point excipient.

The lipid matrix may also include a dispersing agent. In one aspect, the lipid matrix includes from about 0 wt % to about 20 wt %, such as from about 0.01 wt % to about 20 wt %, of a dispersing agent. In another aspect, the lipid matrix includes from about 2 wt % to about 10 wt % of a dispersing agent.

The lipid matrix may also include an antioxidant. In one aspect, the lipid matrix includes from about 0 wt % to about 20 wt %, such as from about 0.01 wt % to about 20 wt %, of an antioxidant. In one aspect, the lipid matrix comprise from about 1 wt % to about 15 wt % of an antioxidant.

The lipid matrix may also include a flow aid. In one aspect, the lipid matrix includes from about 0 wt % to about 5 wt %, such as from about 0.01 wt % to about 5 wt %, of a flow aid. In another aspect, the lipid matrix includes from about 0.5 wt % to about 2 wt % of a flow aid.

In certain aspects, the cores disclosed herein may be subjected to additional processing in order to deposit one or more layers of an active ingredient material on the cores disclosed herein. For example, the cores described herein may contain one or more outer layers disposed on the core that include at least one active ingredient, which may be the same as one or more components in the composition or different from the components in the composition. Further, the one or more outer layers disposed on the core can include an adhesive in addition to one or more active ingredients. For example, to provide the outer layer disposed on the core, the core can be coated with suitable adhesives and then further coated with active ingredients in order to provide a particle having one or more outer layers including one or more active ingredients surrounding the core.

In certain aspects, the one or more particles may be subjected to any suitable coating process for edible tablets or particles in order to apply an active ingredient to the core or to the particles disclosed herein. Suitable processes may include those know generally as the Wurster process. Wurster processes are known in the art and may be synonymous with certain fluid bed microencapsulation processes. Descriptions of Wurster processes are disclosed in U.S. Pat. Nos. 2,648,609 and 3,241,520. Generally, during the Wurster process, the cores or particles are placed in a bed, such as a fluidized bed. The fluidized bed uses differential air flow to create a cyclic movement of the particle material. The one or more cores can be placed in the particulate bed and different air streams can move the bed of particulate material (i.e. the cores) as they are coated with a material, such as a suitable adhesive or active ingredient material, in order to coat the core structure. Generally, the process can be continued until the desired thickness of active ingredient layer is achieved on the core. The particles disclosed herein may be subjected to a suitable Wurster process in order to provide one or more active ingredient layers to the particle. In some embodiments, the cores or particles disclosed herein may be coated via any suitable fluid bed coating process.

In some aspects, the cores and/or particles may be coated via any known drum coating process. Generally, a drum coating process refers to a method for coating particles via placing the particles inside a rotating drum and applying the desired coating material to the particle while rotating the particles within the drum. Devices suitable for drum coating particles are known and include those described in U.S. patent publication no. 2015/0144058. In certain embodiments the cores disclosed herein may be placed in any suitable drum coating device and an adhesive can be applied to the cores or particles. Once sufficiently coated with the adhesive, an active ingredient can then be applied to the cores or particles in the drum to create an active ingredient layer on the cores or particles. This process of applying a suitable adhesive and active ingredient can be repeated as many times as necessary in order to produce the desired number of active ingredient layers on the particles. In certain embodiments, the adhesive may be applied to the core or particle directly with one or more active ingredients.

In one aspect, the cores or particles may be coated with a suitable adhesive. Suitable adhesives may include pharmaceutical grade shellacs such as pharmaceutical glaze, which is an alcohol-based solution that can include various types of food-grade shellac. In certain embodiment, the pharmaceutical glaze may contain from about 20% by weight to about 51% by weight of shellac in an ethyl alcohol solution. The pharmaceutical glaze may further contain additional additives such as waxes, titanium dioxide, and combinations thereof. In certain embodiments, the pharmaceutical glaze utilized according to embodiments herein is certified as generally recognized as safe (GRAS) by the U.S. Food and Drug Administration (FDA). In certain embodiments, the adhesive may include a suitable non-animal-based product, such as zein. Zein generally refers to a class of prolamine protein found in corn that can be manufactured as an adhesive coating or binder.

Accordingly, in some aspects, the outer surface of the cores described herein are coated with a sufficient amount of a suitable adhesive to enable binding of the active ingredient to the outer surface of the core. Generally, the adhesive can be applied such that outer surface of the core becomes tacky or sticky, but is not so tacky such that the cores or particles agglomerate together. Once the adhesive is applied, an active ingredient can be applied to the particle to create an active ingredient layer thereon. In some embodiments, the process of layering the particle with an adhesive layer and layering the particle with an active ingredient can be repeated as many times as desired in order to form a particle having a desired amount of active ingredient layers. For example, the particles disclosed herein may include a core having at least one outer layer thereon containing at least one active ingredient, such as at least two outer layers, such as at least three outer layers, such as at least four outer layers, such as at least five outer layers, such as at least six outer layers, etc. In certain embodiments, each of the outer layers may include one or more active ingredients. In some embodiments, one or more of the outer layers may include the same active ingredient or a different active ingredient from the active ingredient(s) contained within the core. In certain embodiments, the particle may contain one or more outer layers that do not contain an active ingredient. For example, the particle may contain one or more outer layers comprised of a suitable coating material for sealing the particle or for providing a particular release profile upon ingestion.

In some embodiments, the one or more particles provided herein may be formulated into any suitable dosage formulation. For example, in certain aspects, the one or more particles provided herein may be placed into a capsule for delivery by oral ingestion. Exemplary capsules include hard gelatin capsules, soft gelatin capsules, HPMC capsules, as well as capsules made from other materials. The one or more particles may be suspended in an aqueous-based matrix or an oil-based matrix within the capsule itself. In certain embodiments where the particles are suspended in an aqueous-based matrix or an oil-based matrix, the aqueous-based matrix or oil-based matrix may additional include one or more active ingredients. In certain embodiments, the one or more particles may be contained within a monolithic enteric capsule suitable for providing a modified release profile when ingested.

Capsules normally include a shell filled with one or more specific substances. The shell itself may be a soft or a hard capsule shell. Hard capsule shells are generally manufactured using dip molding processes, which can be distinguished into two alternative procedures. In the first procedure, capsules are prepared by dipping stainless-steel mold pins into a solution of polymer, optionally containing one or more gelling agents (e.g. carrageenans) and co-gelling agents (e.g. inorganic cations). The mold pins are subsequently removed, inverted, and dried to form a film on the surface. The dried capsule films are then removed from the molds, cut to the desired length, and then the telescoping fit caps and bodies are assembled together, printed, and packaged. See, e.g., U.S. Pat. Nos. 5,264,223, 5,756,123, and 5,756.123. In the second procedure, no gelling agents or co-gelling agents are used and film-forming polymer solution gelification on the molding pins is thermally induced by dipping pre-heated molding pins into the polymer solution. This second process is commonly referred to as thermogellation, or thermogelling dip molding. See, e.g., EP 0401832, U.S. Pat. Nos. 3,493,407, 4,001,211, and 3,617,588, GB 1310697, and WO 2008/050209. The aforementioned manufacturing processes involve the use of solutions of the different ingredients that are needed for the making the telescoping fit hard capsule shells.

Hard capsules may be filled with active ingredients, such as the composition described herein, via procedures known in the art. Typically, active ingredients (e.g collagen peptide and type II collagen) are combined with various compatible excipients for ease of fill. The resulting fill may be a dry powder, a granulation, particles, lipid particles, a suspension, or a liquid. Additionally, stable, filled hard capsules have advantages over other dosage delivery forms such as liquids and solid tablets. Certain active ingredients may be difficult to formulate into dry granules or may be otherwise incompatible with the tableting process. Another consideration is improved patient compliance for taste-masking and ease of swallowing, i.e., capsules being preferred by consumers over tablets. For example, in some embodiments, provided is a pharmaceutical composition that contains a capsule filled with the one or more particles disclosed herein. In some embodiments, the one or more particles have not been enterically coated for modified release or gastric protection.

Additionally or alternatively, in one aspect, the composition of the present disclosure may be included as an oil-in-water emulsion as a delivery form. Particularly, in one aspect, such an arrangement may allow one or more oil-soluble and/or one or more water-soluble active ingredients to be contained in the same delivery form. Alternatively, only oil-soluble components may be used (e.g. the collagen peptide and the Type II collagen), and the emulsion may be used to incorporate the composition into a water-based application.

Nonetheless, the oil-in-water emulsion may also contain at least one functional gum, such as gum arabic. Gum arabic, in general, is a complex mixture of glycoproteins and polysaccharides, including arabinose and galactose. Gum arabic is generally soluble in water and is edible. In some embodiments, the gum arabic may be comprised of a 100% modified gum arabic, such as Ticamulsion® A-2010 gum arabic powder. In certain embodiments, the gum arabic may be a mixture or blend of gum arabic and modified gum arabic. For example, in certain embodiments, the gum arabic may comprise Ticamulsion® 3020.

In certain aspects, the oil-in-water emulsion contains from about 10% to about 30% by weight of gum arabic. In some embodiments, the oil-in-water emulsion contains from about 15% to about 25% by weight of gum arabic. In some embodiments, the oil-in-water emulsion contains less than about 20% by weight of gum arabic, such as less than 15%, such as less than 10%, such at less than 5%.

The oil-in-water emulsion may also contain water. In certain aspects, the oil-in-water emulsion contains deionized water. Still, in certain aspects, the oil-in-water emulsion may contain any water suitable for human ingestion and incorporation into dietary supplements designed for human ingestion.

The amount of water incorporated into the oil-in-water emulsion can vary depending on the desired hygroscopic and water-soluble ingredients that are incorporated into the oil-in-water emulsion. In certain aspects, the oil-in-water emulsion may contain from about 5% to 35% by weight of water. In some embodiments, the oil-in-water emulsion may contain from about 10% to about 30% by weight of water. In some embodiments, the oil-in-water emulsion may contain from about 15% to about 20% by weight of water. In some embodiments, the oil-in-water emulsion may contain less than about 20% by weight of water, such as less than about 15% by weight of water, such as less than about 10% by weight of water.

In certain aspects, the oil-in-water emulsions may have a water activity of less than about 0.6, such as less than about 0.55, such as less than about 0.5, such as less than about 0.45, such as less than about 0.40, such as less than about 0.35, such as less than about 0.30, such as less than about 0.25, such as less than about 0.20, such as less than about 0.15, such as less than about 0.10. Generally, water activity (aw) described the equilibrium amount of water available for hydration of materials. Water activity generally measures the partial vapor pressure of water in the solution and divides that value by the partial vapor pressure of water at the same temperature to give a final reading. Water activity (aw) can be measured according to the following:

aw=p/p*

For instance, in one aspect, emulsions having water activities over 0.6 may lead to physical instability of the emulsions. Furthermore, it was discovered herein that emulsions having a water activity greater than 0.6 may cause disintegration or dissolve certain HPMC capsules.

In some aspects, the oil-in-water emulsion may contain one or more stabilizers or suspension promoting agents. For example, in certain aspects, the oil-in-water emulsion may contain one or more gum, such as gellan gum or xanthum gum. If included, the gellan gum or xanthum gum may be present in an amount of less than about 3.5% by weight of the oil-in-water emulsion, such as less than about 2.5% by weight, such as less than about 1.5% by weight, such as less than about 1.0% by weight, such as less than about 1.0% by weight.

In other aspects, the oil-in-water emulsion may contain one or more stabilizers such as silica. If included, silica may be present in an amount of less than about 2% by weight, such as less than about 1.5% by weight, such as less than about 1% by weight, such as less than about 0.5% by weight.

Furthermore, in one aspect, the oil-in-water emulsion may also contain one or more fat-soluble ingredients or nutrients. In certain aspects, the one or more fat-soluble ingredients or nutrients may be incorporated into the oil phase of the oil-in-water phase emulsion. Suitable fat-soluble ingredients include, but are not limited to retinol, vitamin E sourced from mixed tocopherols, beta carotene, ubiquinone, lecithin, sunflower lecithin, vitamin D, cannabinoids, hemp extracts, vitamin K, phosphatidyl choline, and combinations thereof.

In certain aspects, at least one or more fat-soluble ingredients may be incorporated in the oil-in-water emulsion in an amount of from about 0% by weight to about 50% by weight. For example, in some aspects, the oil-in-water emulsion contains less than about 50% by weight of one or more fat-soluble ingredients, such as less than about 40% by weight, such as less than about 30% by weight, such as less than about 20% by weight, such as less than about 10% by weight, such a less than about 5% by weight.

Moreover, in one aspect, the oil-in water emulsion may contain one or more additional antioxidants, in one or more of the water soluble phase, or the oil/fat soluble phase.

In some aspects, the oil-in-water emulsion disclosed herein may be provided in any suitable dosage form, such as capsules, tablets, gummy chewables, edible films, lozenges, powders, liquid suspensions, syrups, lipid micelles, spray-dried dispersions, nanoparticles, and the like. In one aspect, the dosage form is an oral dosage form such as a capsule. Capsules are well-known dosage forms that normally consist of a shell filled with one or more specific substances, i.e. the oil-in-water emulsion disclosed herein. The capsule shell may be a soft shell or a hard shell containing film-forming polymers, such as gelatin, modified starches, modified cellulosed, etc.

Furthermore, in an aspect, the oil-in-water emulsion may be formulated to be provided in a capsule. In certain aspects, the capsule may include any suitable HPMC-based capsule. Advantageously, the oral dosage form provided herein may include an HPMC-based capsule containing the oil-in-water emulsion disclosed herein. In one aspect, the HPMC-base capsule may include capsules manufactured by Lonza® Inc. under the tradename of Vcaps®, Vcaps® Plus, or DRcaps®. In some aspects, the capsule may include an modified HPMC-capsule that contains gellan gum and/or thalate. In certain embodiments, the capsule may include an HPMC capsule that has been treated so as to be acid resistant.

In one aspect, the capsule may contain a certain fill weight range or fill volume range. For example, in certain aspects, the capsule may be filled with from about 150 mg to about 1050 mg of the oil-in-water emulsion. In one aspect, the capsule may be filled with about 150 mg of the oil-in-water emulsion. Furthermore, in an aspect, the capsule may be filled with about 20 mg of the oil-in-water emulsion. Moreover, in an aspect, the capsule may be filled with about 280 mg of the oil-in-water emulsion. In some aspects, the capsule may be filled with about 380 mg o more of the oil-in-water emulsion, such as about 500 mg or more of the oil-in-water emulsion, such as about 700 mg or more of the oil-in-water emulsion, such as about 1,050 mg or more of the oil-in-water emulsion.

Alternatively, the oil-in-water emulsion may be contained in a nutritional product, such as a food product or in a beverage. For example, in certain aspects, the oil-in-water emulsion may be incorporated into a liquid nutritional product, such as a nutritional supplement or infant formula, to be consumed by a mammal. Furthermore, the oil-in-water emulsions provided herein may be added to any liquid nutritional product designed to provide nutritional supplementation to a mammal.

Nonetheless, in one aspect, the composition, either alone or as contained in one of the delivery methods above can be administered orally as a solid, liquid, suspension, or gas. The composition may be administered via buccal or sublingual administration. In one embodiment, the composition may be administered as a capsule, tablet, caplet, pill, troche, drop, lozenge, powder, granule, syrup, tea, drink, thin film, seed, paste, herb, botanical, and the like.

Nonetheless, in addition to being administered orally, the composition can also be administered using other routes including intranasal, intravenous, intramuscular, intragastric, and the like.

When the composition, either alone or as part of a delivery form is combined with a food or beverage composition, the food or beverage composition may comprise any suitable composition for consumption by the mammal. Such compositions include complete foods or beverages intended to supply the necessary dietary requirements for mammal or food supplements such as treats and snacks. The food composition may comprise pellets, a drink, a bar, a prepared food contained in a can, a milk shake drink, a juice, a dairy food product, or any other functional food composition. The food composition may also comprise any form of a supplement such as a pill, soft gel, gummy figurine, wafer, or the like.

The composition of the present disclosure may further comprise one or more excipients as further additives in the composition in conjunction with the collagen peptide and the collagen source. Exemplary but non-limiting excipients and/or additives include antiadherents, such as magnesium stearate; binders, such as saccharides, sugar alcohols, gelatin, and synthetic polymers; coatings, such as cellulose ether hydroxypropyl methylcellulose (HPMC), shellac, corn protein zein, gelatin, fatty acids, and waxes; coloring agents, such as titanium oxide and azo dyes; disintegrants, such as modified starch sodium starch glycolate and crosslinked polymers including polyvinylpyrrolidone and sodium carboxymethyl cellulose; fillers, such as maltodextrin; flavoring agents, such as mint, liquorice, anise, vanilla, and fruit flavors including peach, banana, grape, strawberry, blueberry, raspberry, and mixed berry; glidants, such as fumed silica, talc, and magnesium carbonate; lubricants, such as talc, silica, and fats including vegetable stearin, magnesium stearate, and stearic acid; preservatives, such as antioxidants, vitamins, retinyl palmitate, selenium, the amino acids cysteine and methionine, citric acid, sodium citrate, and parabens; sorbents; sweeteners, such as sucrose and sucralose; and vehicles, such as petrolatum and mineral oil.

In one aspect, the composition of the present disclosure may be combined with various additives and components that can improve one or more properties of the composition. For example, in one embodiment, the additive composition may be combined with a stabilizer package that may serve to stabilize at least one property of the composition. In one particular embodiment, for instance, a stabilizer package may be added to the composition in an amount sufficient to reduce the hydroscopic properties of the composition and/or prevent the composition from absorbing moisture. A stabilizer package may also be combined with the composition in order to improve the handling properties of the composition. For instance, the stabilizer package may allow the composition to have better flow properties, especially when in granular form.

In one aspect, the composition may be combined with a polymer binder in conjunction with a stabilizer package. In addition, a coating material may also be applied to the composition after the composition has been combined with the polymer binder and the stabilizer package. The coating material, for instance, may contain at least one fat. In accordance with the present disclosure, the above components can be added to any suitable pharmaceutical composition in addition to the composition of the present disclosure. For instance, the above components may be added to any pharmaceutical composition containing a carnitine or an amino acid.

The polymer binder and the stabilizer package may be combined with the composition in a manner that homogeneously incorporates the stabilizer package into the product. In one embodiment, for instance, the composition of the present disclosure is first combined with a polymer binder, such as through a spray dry process, and then combined with the stabilizer package. The polymer binder may comprise any suitable pharmaceutically acceptable polymer, such as film-forming polymers and/or polysaccharides. Particular examples of polymer binders that may be used in accordance with the present disclosure include starch, maltodextrin, gum arabic, arabinogalactan, gelatin, and mixtures thereof. In one embodiment, the polymer binder is added to the pharmaceutical composition in an amount of at least about 5% by weight, such as at least about 8% by weight, such as at least about 10% by weight, such as at least about 15% by weight. One or more polymer binders are present in the composition in an amount less than about 50% by weight, such as in an amount less than about 45% by weight, such as in an amount less than about 40% by weight, such as in an amount less than about 35% by weight, such as in an amount less than about 30% by weight.

In one embodiment, the polymer binder may comprise a starch, such as a modified starch. The starch, for instance, may be derived from corn or waxy maize. In one embodiment, the starch may comprise HI-CAP100 starch sold by National Starch and Chemical Company.

In an alternative embodiment, the polymer binder may comprise arabinogalactan. Arabinogalactan is a soluble polysaccharide that not only can serve as a polymer binder but may also provide other benefits. For instance, arabinogalactan may enhance the adaptive immune response in some circumstances. Arabinogalactan is described, for instance, in U.S. Pat. No. 8,784,844, which is incorporated herein by reference.

In one embodiment, larch arabinogalactan may be used as the polymer binder. Larch arabinogalactan is a highly branched polysaccharide that is composed of galactose units and arabinose units in the approximate ratio of 6:1. Larch arabinogalactan is extracted from large trees. The polysaccharide has a galactan backbone with side chains of galactose and arabinose. Arabinogalactan is commercially available from Lonza Ltd.

Once the polymer binder is combined with the composition such as through a spray dry process, the resulting mixture can then be combined with a stabilizer package. In one embodiment, the stabilizer package comprises oxide particles in combination with a salt of a carboxylic acid. In one particular embodiment, the stabilizer package may comprise a dry product, such as a powder or granular product that is combined with the composition and polymer binder. The combination of oxide particles and a salt of a carboxylic acid have been found to provide numerous advantages and benefits when combined with the composition. For instance, the stabilizer package has been found to stabilize the composition and make the composition less hydroscopic. The composition is also easier to handle and, when in granular form, produces a free-flowing product.

The oxide particles that may be added to the pharmaceutical composition may comprise silica. For instance, the oxide particles may comprise precipitated silica particles. The silica particles may have a particle size (d50, laser defraction following ISO Test 13320) of less than about 55 microns, such as less than about 40 microns, such as less than about 30 microns, such as less than about 25 microns, such as less than about 20 microns, such as less than about 15 microns, such as less than about 12 microns, such as less than about 10 microns, such as less than about 8 microns, such as less than about 6 microns, such as less than about 4 microns, such as less than about 2 microns, such as less than about 1 micron. The particle size is typically greater than about 0.5 microns, such as greater than about 1 micron. The particles may have a specific surface area (ISO Test 9277) of greater than about 120 m2/g, such as greater than about 130 m2/g, such as greater than about 150 m2/g, such as greater than about 170 m2/g, such as greater than about 200 m2/g, such as greater than about 220 m2/g. The specific surface area is generally less than about 500 m2/g. The oxide particles, such as the silica particles, can be present in the pharmaceutical composition in an amount greater than about 0.01% by weight, such as in an amount greater than about 0.05% by weight, such as in an amount greater than about 0.1% by weight. The oxide particles are generally present in an amount less than 5% by weight, such as in an amount less than about 2% by weight, such as in an amount less than about 1.5% by weight, such as in an amount less than 0.5% by weight.

In addition to the oxide particles, the stabilizer package may also include a salt of a carboxylic acid. The salt of a carboxylic acid may comprise a salt of a fatty acid. The fatty acid, for instance, may have a carbon chain length of from about 6 carbon atoms to about 40 carbon atoms, such as from about 12 carbon atoms to about 28 carbon atoms. In one embodiment, the salt of the carboxylic acid may comprise a stearate salt. The stearate salts that may be used include calcium stearate, sodium stearate, magnesium stearate, mixtures thereof, and the like. In one embodiment, the salts of the carboxylic acid may include both hydrophilic groups and hydrophobic groups. The salt of the carboxylic acid may be present in the composition in an amount greater than about 0.5% by weight, such as in an amount greater than about 1% by weight, such as in an amount greater than about 1.5% by weight. The salt of the carboxylic acid is generally present in an amount less than about 5% by weight, such as in an amount less than about 4% by weight, such as in an amount less than about 3% by weight.

In addition to the polymer binder and the stabilizer package, the composition may include various other components and ingredients. In one embodiment, for instance, the composition may contain a citric acid ester, such as a citric acid ester of a mono and/or diglyceride of a fatty acid. The composition may also contain a lecithin, such as a lecithin obtained from rapeseed, sunflower, and the like. The above components can be present in the composition in relatively minor amounts, such as less than about 2% by weight, such as less than about 1.5% by weight, such as less than about 1% by weight. The above components are generally present in an amount greater than about 0.05% by weight, such as in an amount greater than about 0.1% by weight.

Once the above components are combined together to form the composition, the composition can optionally be combined with a coating material. In one embodiment, for instance, the composition may comprise a granular composition to which a coating material is applied that contains a fat. The coating material, for instance, may comprise a hydrogenated oil, such as hydrogenated palm oil. In one particular embodiment, the coating material may comprise hydrogenated palm oil combined with palm stearine. In one embodiment, the hydrogenated oil may be present in the pharmaceutical composition in an amount from about 5% to about 35% by weight. The palm stearine, on the other hand, may be present in the pharmaceutical composition in an amount from about 2% to about 10% by weight. When present together, the weight ratio between the hydrogenated palm oil and the palm stearine may be from about 10:1 to about 1:1, such as from about 6:1 to about 2:1. In one embodiment, the hydrogenated palm oil and the palm stearine are present at a weight ratio of about 4:1.

In one aspect, the composition can be also be incorporated into a topical composition that is intended to be topically applied to the skin of a user. The composition may be incorporated into the topical composition in an amount of about 1% to about 25% by weight of the topical composition, such as about 5% to about 20% by weight, such as about 7.5% to about 20% by weight, or any ranges or values therebetween. When formulated as a topical composition, for instance, the collagen source and collagen peptide can be blended with various ingredients and components. For instance, when formulated as a topical composition, the composition of the present disclosure can be blended with solvents, surfactants, emulsifiers, consistency factors, conditioners, emollients, skin caring ingredients, moisturizers, thickeners, lubricants, preservatives, and various different dermatological ingredients. When applied to a skin of a user, the composition of the present disclosure can be used to improve skin health or can be formulated so as to be absorbed into the body.

Suitable emulsifiers for a topical composition may include a polyglyceryl fatty ester, an ethoxylated fatty alcohol, an ethoxylated fatty acid, an ethoxylated sorbitan ester, mixtures thereof, and the like.

Polyglyceryl esters useable in a topical composition may be formed from saturated, unsaturated, natural or synthetic fatty acids, mixtures thereof, and the like. For instance, examples of saturated fatty acids include, but are not limited to, caprylic acid, capric acid, lauric acid, myristic acid, palmitic acid, stearic acid, arachidic acid, behenic acid, lignoceric acid, cerotic acid, oleic acid, decaoleic acid, combinations thereof, derivatives thereof, and the like.

Furthermore, the polyglyceryl esters may be derived from (a) a polyglycerol component built up from 2 to 12 molecules of glycerol, based on an average, and (b) a fatty acid comprising of a caprylic acid, capric acid, lauric acid, myristic acid, palmitic acid, stearic acid, arachidic acid, behenic acid, lignoceric acid, cerotic acid, oleic acid, decaoleic acid, or mixtures thereof, and the like.

When used, an emollient can be a wax, oil, mixtures thereof, and the like. In one aspect, the emollient is a wax or an oil, whereby the wax or oil is present in an amount between 15 w/w %-80 w/w %; based on the weight of the composition. Suitably the emollient is present in an amount between 40 w/w %-80 w/w %; based on the weight of the composition.

The term “waxes” herein, refers to naturally-occurring or synthetic waxes, of which typical examples include stearyl alcohol, hydrocarbon waxes, waxes of plant or animal origin, their synthetic analogues or derivatives, or silicone waxes. Waxes are widely available, and by suitable selection of the waxes themselves and their concentrations in the formulation can effectively obtain either a soft solid or a firm solid. Conventionally, waxes are applied to a variety of materials and mixtures which have similar physical properties, namely that: they are solid at about 30° C. to about 40° C.; they melt to a mobile liquid at a temperature above 30° C., and below 95° C.; generally in a temperature range of 40° C. to about 90° C.; and they are water-insoluble and remain water-immiscible when heated above their melting point.

A variety of waxes may be suitable, including but not limited to, silicone polymers, hydrocarbons, linear fatty alcohols, esters of fatty acids or glyceride derivatives, mixtures thereof, or complexes thereof, and the like.

Examples of waxes for soft solid/stick applications include, but are not limited to, castor wax, beeswax, carnauba and candelilla waxes, which are of vegetable origin and mineral waxes from fossil remains other than petroleum. Montan wax, which is an example of mineral wax, includes non-glyceride esters of carboxylic acids, hydrocarbons and other constituents. Other naturally available waxes include spermeceti wax, ozokerite, ceresin, baysberry, synthetic waxes, paraffin wax, silicone waxes or mixtures thereof, and the like.

The term “oils” herein, refers to an organic compound which at about 20° C. is both liquid and water-insoluble. In the context of the invention, insolubility in water is understood to be a solubility of less than 10 w/w % at about 20° C. Typically, the solubility of less than 1 w/w %, more particularly less than 0.1 w/w %, in particularly less than 0.01 w/w %.

Examples of oil emollients for a topical composition include, but are not limited to, ester oils, an ether oil or a mixture thereof, and the like. The term “ester oil” herein, refers to oils as above-defined, which comprises at least one ester group. This means that also esters of carbonic acids are ester oils according to the invention. Further, the term “ether oil” herein, refers to oils as above-defined, which comprises at least one ether group.

Examples of oils for a topical composition include, but are not limited to, glycerides (such as e.g., triglycerides), hydrocarbons (such as e.g., petrolatum); silicone oils (such as e.g., dimethicone), dialkyl ethers, alkyl esters, dialkyl carbonates, dialkyl tartrates, natural oils (such as vegetable oils), and the like. In dialkyl ethers, alkyl esters and dialkyl carbonates such as dicaprylyl carbonate, ethylhexyl carbonate, dihexyl carbonate; the alkyl groups may be straight or branched and independently from each other typically have C2-C16 atoms. Generally, at least one of said alkyl groups has at least 6 carbon atoms, typically at least 8 carbon atoms. Optionally the oil may be an oil that has further functions such as an oil soluble sunscreen. Mixtures of oil components may also be used.

Typically, the emollient includes, but is not limited to, a dialkyl ether such as dicaprylyl ether, or an alkyl ester, C12-C15 alkyl benzoates or C10-C16 dialkyl tartrates. Generally, the emollient is a C10-C16 dialkyl tartrate, specifically C12-C13 dialkyl tartrate; or a dicaprylyl ether, specifically dicaprylyl carbonate.

Suitably, emollients include, but are not limited to, C12-C13 dialkyl tartrate, dicaprylyl carbonate, triC12-C13 tartrate, marula oil, sunflower seed oil (Helianthus annuss), safflower seed oil (Carthamus tinctorius), coconut oil, capryl/caprylic triglyceride, linseed oil, canola oil, cottonseed oil, soybean oil, or mixtures thereof, and the like.

A topical composition may also contain one or more preservative including, but is not limited to, benzoic acid or salt thereof, benzyl alcohol, sorbic acid or salt thereof, parabens, dehydroacetic acid, sodium dehydroacetate, bronopol, triclosan, imidazolidinyl urea, PHMB (polyhexamethylene biguanide), phenoxyethanol, DMDMH (1,3-Dimethylol-5,5-dimethylhydantoin), isothiazolones, chlorhexidine, diazolidinyl urea, chlorphenesin, sodium hydroxymethylglycinate, benzethonoium chloride, ethylhexylglycerol, IPBC (iodopropynyl butylcarbamate), salicylic acid or salt thereof, sodium benzoate, calcium gluconate, D-glucono1,5-lactone, glycerin, or natural acid or any preservative from the Annex V preservative list, and the like.

Typically the preservative system is present in a topical composition in an amount between 0.01 w/w %-10 w/w %; whereby the total amount of all preservatives used is within the provided range; based on the total weight of the composition. Suitably, the preservative system is present in an amount between 0.5 w/w %-5 w/w %, based on the total weight of the composition.

Suitably, the preservative system includes, but not limited to, one or more of a dehydroacetic acid, benzyl alcohol, sorbic acid, salicyclic acid, benzoic acid, sodium benzoate, calcium gluconate, D-glucono1,5-lactone, glycerin, phenoxyethanol, DMDMH, sodium dehydroacetate, iodopropynyl butylcarbamate (IPBC) and the like.

Typically the aqueous solvent is present in a topical composition in an amount wherein the concentrate composition totals up to 100 w/w %, based on the weight of the composition. Suitably, the aqueous solvent is present in an amount between 15w/w %-55 w/w %.

Examples of aqueous solvents for a topical composition include, but not limited to, water, aqueous alcohols, ammonia water, acid solutions, salt solutions, water-miscible organic solvents, alkanolamines, glycol ethers, polyhydroxy alcohols (such as glycerin, diglycerin, propylene glycol, dipropylene glycol, sorbitol, pantenol and sugar); urea, alpha-hydroxy acid and its salt; and low molecular weight polyethylene glycols with molecular weight less than 20,000; water soluble or dispersible polymers; alkanolamines, or glycol ethers or mixtures thereof; and the like. Generally, the solvent used is water or an aqueous alcohol.

The term “aqueous alcohol” herein, refers to saturated, unsaturated, straight or branched hydrocarbon chain having C2-C4 carbon atoms and at least one hydroxyl groups, wherein the hydrocarbon chain may optionally comprise one or several heteroatoms (such as oxygen or sulfur).

Although not necessary, humectants may be added to a topical composition, if desired, without disturbing the properties within the cleansing concentrate composition. Suitable humectants useable as component can essentially be any known humectant that will not cloud or discolor the concentrate.

Examples of humectants for topical compositions include, but are not limited to, amino acids, pyrrolidone carboxylic acid, lactic acid and salts thereof, lactitol, urea and urea derivatives, uric acid, glucosamine, creatinine, cleavage products of collagen, chitosan or chitosan salts/derivatives and, in particular, polyols and polyol derivatives, for example glycerol, diglycerol, triglycerol, polyglyerin, such as polyglycerin-6, ethylene glycol, propylene glycol, butylene glycol, erythritol, 1,2,6-hexanetriol, polyethylene glycols, sugars and sugar derivatives (e.g. fructose, glucose, maltose, maltitol, mannitol, inositol, sorbitol, sorbitol silanediol, sucrose, trehalose, xylose, xylitol, glucuronic acid and salts thereof), ethoxylated sorbitol (Sorbeth-6, Sorbeth-20, Sorbeth-30, Sorbeth-40), honey and hydrogenated honey, hydrogenated starch hydrolyzates and mixtures of hydrogenated wheat protein and PEG-20-acetate copolymer, and the like.

Accordingly, if a humectant is desired, the humectant may be a polyol, in particular propylene glycol, glycerol, diglycerol and/or triglycerol. Generally, the humectant may be present in an amount between 10 w/w %-45 w/w %, based on the weight of the composition; or the humectant may be present in an amount <10 w/w %, based on the weight of the composition. Furthermore, the cleaning concentrate composition may be essentially free of a humectant.

Nonetheless, certain embodiments of the present disclosure may be better understood according to the following proposed example, which are intended to be non-limiting and exemplary in nature.

### PROPOSED EXAMPLES

### Example 1

Testing Rat Model: Collagen supplementation against UV-Induced Skin damage by using a study period of 8 to 12 weeks using UVB photoaging of the skin in shaved rats and unshaved rats. Various formulations of 20 mg of undenatured collagen (equivalent human dose) with various amounts of collagen protein will be oral and topically given to the rats. Study will Evaluation of Dorsal Skin, Biochemical Analysis-Skin tissues, Dorsal skin protein levels, Histology and immunohistochemistry, Macroscopic Appearance-dorsal skin lesions caused by UVB Exposure & improvement with supplementation and last skin elasticity.

### Example 2

UV Induced-Photoaging on the Hair Follicle Cycle of C57BL6/J Mice. by using a study period of 8 to 12 weeks using UVB photoaging of the skin in shaved mice and unshaved mice. Various formulations of 20 mg of undenatured collagen (equivalent human dose) with various amounts of collagen protein will be oral and topically given to the rats. Changes in skin and the hair follicle cycle will be compared by physical signs, dermoscopy, and hematoxylin and eosin and Masson's staining in each group. Western blot, immunohistochemistry, and RT-qPCR are to be carried out to test canonical proteins and gene expression of the Wnt signaling pathway in the samples. Immunofluorescence chosen to show variations in the stem cells related to the hair follicle cycle.

These and other modifications and variations to the present invention may be practiced by those of ordinary skill in the art, without departing from the spirit and scope of the present invention, which is more particularly set forth in the appended claims. In addition, it should be understood that aspects of the various embodiments may be interchanged both in whole or in part. Furthermore, those of ordinary skill in the art will appreciate that the foregoing description is by way of example only, and is not intended to limit the invention so further described in such appended claims.

What is claimed:

1. A method of improving skin health of a mammal, comprising:
administering a composition comprising a native or undenatured type II collagen and a collagen peptide to a healthy mammal in an amount sufficient to improve one or more of skin tone and elasticity, overall skin hydration, photoaging, skin microbiome, and wrinkle reduction,
wherein the type II collagen is present in the composition in an amount from about 1 mg to about 1000 mg; and
wherein the collagen peptide is in the composition in an amount of 1 mg to about 10 g.

2. The method of claim 1, wherein collagen peptide is present in the composition in an amount of about 100 mg to about 1000 mg.

3. The method of claim 1, wherein the composition is administered daily.

4. The method of claim 1, wherein the composition further includes at least one of a vitamin, a mineral, an essential fatty acid, or an amino acid.

5. The method of claim 4, wherein compositions includes a vitamin and the vitamin comprises vitamin E, vitamin C, or a combination thereof.

6. The method of claim 4, wherein the composition includes potassium, magnesium, calcium, zinc, or combinations thereof.

7. The method of claim 1, wherein the improvement in skin health is evidenced by a reduction in wrinkles or skin irregularities of about 5% or greater, as evaluated by image analysis.

8. The method of claim 1, wherein the improvement in skin health is evidenced by an improvement in skin flaccidness, where the mammal shows an improvement in v-shaped analysis of about 5% or greater, as measured by image analysis.

9. The method of claim 1, wherein the improvement in skin health is evidenced by an improvement in skin moisture of about 5% or greater, as measured using electrical capacitance.

10. The method of claim 1, wherein the improvement in skin health is evidenced by an improvement in skin elasticity and/or firmness of at least about 5%, as measured using a Cutometer.

11. The method of claim 1, wherein the native or undenatured type II collagen is present in the composition in an amount of about 5 mg to about 500 mg, for example about 15 mg to about 200 mg, in particular 20 mg to about 100 mg.

12. A composition for improving skin health of a mammal, comprising:
a type II collagen present in an amount from about 1 mg to about 1000 mg; and
a collagen peptide is present in the composition in an amount of 1 mg to about 10 g.

13. The composition of claim 12, wherein the type II collagen includes native type II collagen and undenatured type II collagen.

14. The composition of claim 13, wherein the undenatured type II collagen is present in the composition in an amount of about 1 mg to about 100 mg.

15. The composition of claim 12, wherein the collagen peptide is present in the composition in an amount of about 100 mg to about 1000 mg.

16. The composition of claim 12, wherein the collagen peptide is present in the composition in an amount of about 100 mg to about 1000 mg and the wherein the type II collagen includes native type II collagen and undenatured type II collagen is present in an amount of about 1 mg to about 100 mg.

17. The composition of claim 12, further comprising least one of a vitamin, a mineral, an essential fatty acid, or an amino acid.

18. The composition of claim 12, wherein the composition is formulated into one or more multiparticulate dosage forms.

19. The composition of claim 12, wherein the composition is coated on a core, contained in a lipid multiparticulate, or contained in an oil-in-water emulsion.

20. The composition of claim 12, wherein the one or more multiparticulate dosage forms is suitable for incorporation into a capsule, a beverage, a food product, a topical formulation, or combinations thereof.
